(19) United States
(12) Patent Application Publication
(10) Pub. No.: US 2010/0292179 A1
(43) Pub. Date: Nov. 18, 2010

(54) TARGET

(75) Inventors: Paul William Denny, Middlesbrough (GB); Ralph Thomas Schwarz, Marburg (DE); Hosam Shams-Eldin, Marburg (DE)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: The University of Durham, Durham (UK)

(21) Appl. No.: 12/161,523

(22) PCT Filed: Jan. 19, 2007
(86) PCT No.: PCT/GB2007/000142

§ 371 Date: Jul. 19, 2010

(30) Foreign Application Priority Data
Jan. 19, 2006 (GB) 0601059.9

(51) Int. Cl. A61K 31/7028; G01N 33/50; C12N 9/00; C12N 5/10; C12N 1/15; C12N 1/19; A61K 31/407; A61K 31/164; C07G 11/00; C07D 267/22; C07C 233/02; A61P 31/04; A61P 31/10; A61P 33/02; G06G 7/58

(52) U.S. Cl. 514/28; 435/7.1; 435/183; 435/325; 435/348; 435/418; 435/254.11; 435/254.2; 435/254.21; 435/7.21; 435/7.31; 435/7.2; 514/410; 514/625; 536/16.8; 540/455; 564/192; 703/11

(57) ABSTRACT

We describe the functional cloning and the use of a non-fungal inositol phosphoryceraminde synthase (IPC synthase) in a screening assay for the identification of agents that target and antagonize the activity of IPC synthase.

The invention relates to the use of a non-fungal inositol phosphoryceraminde synthase polypeptide (IPC synthase) in a screening assay for the identification of agents that target and antagonize the activity of IPC synthase.

The evolutionarily divergent, insect vector borne protozoan parasites of the order Kinetoplastidae cause a range of human diseases, including the leishmaniases (a broad spectrum of infections caused by Leishmania species), Chagas' disease (Trypanosoma cruzi) and African sleeping sickness (T. brucei). These infections are of increasing prevalence in developing countries where mortality is often affected by poor access to health care. Many of the drugs available to treat these diseases are expensive, difficult to administer or toxic and no effective vaccines are available. These facts make the discovery of new anti-kinetoplastid therapeutic targets and compounds of paramount importance.

Sphingolipids are ubiquitous and essential components of eukaryotic membranes, particularly the plasma membrane. The biosynthetic pathway for the formation of these lipid species is conserved up to the formation of sphinganine. However, a divergence is apparent in the synthesis of complex sphingolipids. In animal cells ceramide is a substrate for sphingomyelin (SM) production via the enzyme SM synthase. In contrast, fungi utilize phytoceramide in the synthesis of inositol phosphorylceramide (IPC) catalyzed by IPC synthase. Due to the absence of a mammalian equivalent, this essential enzyme represents an attractive target for anti-fungal compounds.

Fungal IPC synthase genes have been cloned and characterized. US2003022342 describes the cloning of a variety of IPC synthase genes from fungal species and the use of fungal IPC synthase in screening assays to identify anti-fungal agents. U.S. Pat. No. 6,307,037 describes further IPC synthase genes from the fungus Ashbya gosssypii. These have also been utilized in screening assays for the identification of anti-fungal agents. EP1131449 describes a cell-based assay for use in the identification of anti-fungal agents that specifically inhibit IPC synthase. The assay utilizes fungal cells, in particular Saccharomyces cerevisae cells, that are mutated in a gene (LCB-1 gene) that prevents the synthesis of sphingolipids. The strain also includes a suppressor of the Icb-1 mutation called SLC1-1 and is viable but grows poorly except in the presence of phytosphingosine. The cells provide a sensitive means to detect agents that inhibit fungal IPC synthase. Anti-fungal agents that act by inhibition of IPC synthase are known and are natural antibiotics such as aureobasidin, khafrefungin and rustmicin.

In common with the fungi, the kinetoplastid protozoa and higher plants synthesize IPC rather than SM. However, orthologues of the fungal IPC synthase are not readily identified in the complete genome databases of these species. We have isolated the gene encoding IPC synthase in the kinetoplastids, causative agents of a range of important human diseases. This gene product, that can fully constitute enzyme activity and be specifically inhibited by an anti-fungal targeting IPC synthase defines a new class of protozoan sphingolipid synthases. The identification and characterization of the protozoan IPC synthase, an enzyme with no mammalian equivalent, raises the possibility of developing anti-protozoan drugs with minimal toxic side-affects.

In addition we have isolated plant homologues of the protozoan IPC synthase that has similar characteristics to the protozoan enzyme and therefore provides an opportunity to screen for agents that inhibit the plant enzyme. These are potentially useful as herbicides.

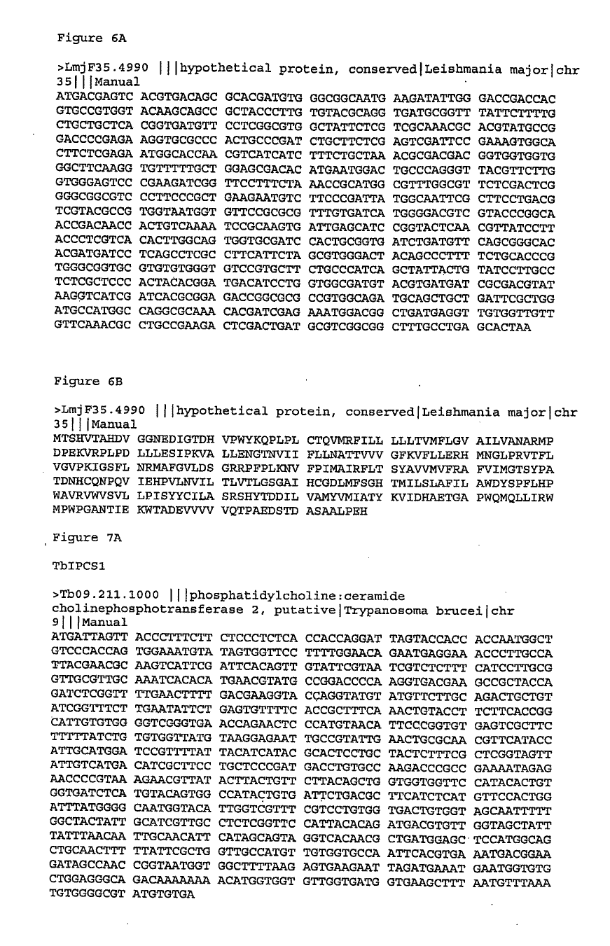

According to an aspect of the invention, there is provided a polypeptide encoded by a nucleic acid molecule selected from the group consisting of:
i) a nucleic acid molecule consisting of the DNA sequence as represented in FIG. 6A, 7A, 8A, 9A, 10A, 11A, 12A, 13A, 14A or 15A;
ii) a nucleic acid molecule comprising DNA sequences that hybridize to the sequence identified in (i) above under stringent hybridization conditions and which encodes a polypeptide that is an inositol phosphorylceramide synthase; and
iii) a nucleic acid molecule comprising DNA sequences that are degenerate as a result of the genetic code to the DNA sequence defined in (i) and (ii), and encodes a polypeptide that is an inositol phosphorylceramide synthase.

Preferably, said polypeptide comprises the amino acid sequence as shown in FIG. 7B, 8B, 9B, 10B, 11B, 12B, 13B, 14B or 15B, or sequence variant thereof, wherein said variant is modified by deletion, addition or substitution of at least one amino acid residue. Preferably, said polypeptide consists of the amino acid sequence shown in FIG. 7B, 8B, 9B, 10B, 11B, 12B, 13B, 14B or 15B.

Preferably, said nucleic acid molecule has at least 50% sequence identity, over all or part of the length of the nucleic acid, to the nucleic acid as represented by the nucleic acid sequence in FIG. 6A, 7A, 8A, 9A, 10A, 11A, 12A, 13A, 14A or 15A and which encodes a polypeptide that has inositol phosphorylceramide synthase activity.

Preferably, said polypeptide is a variant polypeptide and comprises the amino acid sequence represented in FIG. 7B, 8B, 9B, 10B, 11B, 12B, 13B, 14B or 15B, which sequence has been modified by deletion, addition or substitution of at least one amino acid residue wherein said modification retains or modifies the inositol phosphorylceramide synthase activity of said polypeptide.

According to an aspect, there is provided a cell wherein said cell is transformed or transfected with a nucleic acid molecule that encodes a polypeptide according to the invention.

According to an aspect of the invention there is provided a cell wherein said cell is transformed or transfected with a nucleic acid molecule comprising a nucleic acid sequence selected from the group consisting of:
i) a nucleic acid molecule consisting of the DNA sequence as represented in FIG. 6A, 7A, 8A, 9A, 10A, 11A, 12A, 13A, 14A or 15A;
ii) a nucleic acid molecule comprising DNA sequences that hybridize to the sequence identified in (i) above under stringent hybridization conditions and which encodes a polypeptide that is an inositol phosphorylceramide synthase; and
iii) a nucleic acid molecule comprising DNA sequences that are degenerate as a result of the genetic code to the DNA sequence defined in (i) and (ii), and encodes a polypeptide that is an inositol phosphorylceramide synthase.

Hybridization of a nucleic acid molecule occurs when two complementary nucleic acid molecules undergo an amount of hydrogen bonding to each other. The stringency of hybridization can vary according to the environmental conditions surrounding the nucleic acids, the nature of the hybridization method, and the composition and length of the nucleic acid molecules used. Calculations regarding hybridization conditions required for attaining particular degrees of stringency are discussed in Sambrook et al., Molecular Cloning: A Laboratory Manual (Cold Spring Harbor Laboratory Press, Cold Spring Harbor, N.Y., 2001); and Tijssen, Laboratory Techniques in Biochemistry and Molecular Biology—Hybridization with Nucleic Acid Probes Part I, Chapter 2 (Elsevier, N.Y., 1993). The Tₘ is the temperature at which 50% of a given strand of a nucleic acid molecule is hybridized to its complementary strand. The following is an exemplary set of hybridization conditions and is not limiting:

Very High Stringency (allows sequences that share at least 90%
identity to hybridize)

| | |
|---|---|
| Hybridization: | 5x SSC at 65° C. for 16 hours |
| Wash twice: | 2x SSC at room temperature (RT) for 15 minutes |
| | each |
| Wash twice: | 0.5x SSC at 65° C. for 20 minutes each |

High Stringency (allows sequences that share at least 80%
identity to hybridize)

| | |
|---|---|
| Hybridization: | 5x-6x SSC at 65° C.-70° C. for 16-20 hours |
| Wash twice: | 2x SSC at RT for 5-20 minutes each |
| Wash twice: | 1x SSC at 55° C.-70° C. for 30 minutes each |

Low Stringency (allows sequences that share at least 50%
identity to hybridize)

| | |
|---|---|
| Hybridization: | 6x SSC at RT to 55° C. for 16-20 hours |
| Wash at least twice: | 2x-3x SSC at RT to 55° C. for 20-30 minutes each. |

In a preferred embodiment of the invention said nucleic acid molecule has at least 20% sequence identity, over all or part of the length of the nucleic acid, to the nucleic acid as represented by the nucleic acid sequence in FIG. 6A, 7A, 8A, 9A, 10A, 11A, 12A, 13A, 14A or 15A and which encodes a polypeptide that has inositol phosphorylceramide synthase activity.

In addition, the invention features nucleic acid molecules comprising nucleic acid sequences having at least 30% identity with the nucleic acid sequences as herein disclosed. In one embodiment, the nucleic acids have at least 40% identity, more preferably at least 50% identity, even more preferably at least 60% identity, still more preferably at least 70%, more preferably 80% identity, and most preferably at least 90% identity with the nucleic acid sequences illustrated herein.

In a preferred embodiment of the invention said nucleic acid molecule encodes a polypeptide that is represented by the amino acid sequence in FIG. 7B, 8B, 9B, 10B, 11B, 12B, 13B, 14B or 15B.

In a further preferred embodiment of the invention said polypeptide is a variant polypeptide and comprises the amino acid sequence represented in FIG. 7B, 8B, 9B, 10B, 11B, 12B, 13B, 14B or 15B, which sequence has been modified by deletion, addition or substitution of at least one amino acid residue wherein said modification retains or modifies the inositol phosphorylceramide synthase activity of said polypeptide.

A variant polypeptide may differ in amino acid sequence by one or more substitutions, additions, deletions, truncations that may be present in any combination. Among preferred variants are those that vary from a reference polypeptide by conservative amino acid substitutions. Such substitutions are those that substitute a given amino acid by another amino acid of like characteristics. The following non-limiting list of amino acids are considered conservative replacements (similar): a) alanine, serine, and threonine; b) glutamic acid and aspartic acid; c) asparagine and glutamine d) arginine and lysine; e) isoleucine, leucine, methionine and valine and f) phenylalanine, tyrosine and tryptophan. Most highly preferred are variants that retain or modify the same biological function and activity as the reference polypeptide from which it varies.

In addition, the invention features polypeptide sequences having at least 50% identity, over all or part of the length of the polypeptide, with the polypeptide sequences as herein disclosed, or fragments and functionally equivalent polypeptides thereof. In one embodiment, the polypeptides have at least 60% identity, more preferably at least 70% identity, even more preferably at least 80% identity, still more preferably at least 90% identity, and most preferably at least 99% identity with the amino acid sequences illustrated herein.

In a preferred embodiment of the invention said nucleic acid molecule is part an expression vector.

A vector including nucleic acid (s) according to the invention need not include a promoter or other regulatory sequence, particularly if the vector is to be used to introduce the nucleic acid into cells for recombination into the genome for stable transfection. Preferably the nucleic acid in the vector is operably linked to a suitable promoter or other regulatory elements for transcription in a host cell. The vector may be a bi-functional expression vector which functions in multiple hosts. By “promoter” is meant a nucleotide sequence upstream from the transcriptional initiation site and which contains all the regulatory regions required for transcription. Suitable promoters include constitutive, tissue-specific, inducible, developmental or other promoters for expression in cells. Such promoters include viral, fungal, bacterial, animal and plant-derived promoters. “Operably linked” means joined as part of the same nucleic acid molecule, suitably positioned and oriented for transcription to be initiated from the promoter. DNA operably linked to a promoter is “under transcriptional initiation regulation” of the promoter.

In a preferred embodiment the promoter is a constitutive, an inducible or regulatable promoter.

In a further preferred embodiment of the invention said cell is selected from the group consisting of a fungal cell; insect cell; a mammalian cell; a plant cell.

In a preferred embodiment of the invention said cell is preferably a fungal cell. Preferably said cell is a yeast cell, for example a Saccharomyces cerevisae cell.

According to a further aspect of the invention there is provided the use of a polypeptide encoded by a nucleic acid molecule selected from the group consisting of
i) a nucleic acid molecule consisting of the DNA sequence as represented in FIG. 6A, 7A, 8A, 9A, 10A, 11A, 12A, 13A, 14A or 15A;
ii) a nucleic acid molecule comprising DNA sequences that hybridize to the sequence identified in (i) above under stringent hybridization conditions and which encodes a polypeptide that is an inositol phosphorylceramide synthase; and
iii) a nucleic acid molecule comprising DNA sequences that are degenerate as a result of the genetic code to the DNA sequence defined in (i) and (ii), and encodes a polypeptide that is an inositol phosphorylceramide synthase.

According to a further aspect, there is provided the use of a polypeptide according to the invention for the identification of an agent that modulates the activity of an inositol phosphoryceraminde synthase enzyme.

According to a further aspect, there is provided the use of a cell according to the invention for the identification of an agent that modulates the activity of an inositol phosphoryceraminde synthase enzyme.

In a preferred embodiment said modulation is inhibition of an inositol phosphoryceraminde synthase enzyme

According to a yet further aspect of the invention there is provided a screening Method for the identification of an agent that has inositol phosphorylceramide synthase enzyme inhibitory activity comprising the steps of:
i) providing a polypeptide encoded by a nucleic acid molecule selected from the group consisting of:
   a) a nucleic acid molecule consisting of the DNA sequence as represented in FIG. 6A, 7A, 8A, 9A, 10A, 11A, 12A, 13A, 14A or 15A;
   b) a nucleic acid molecule comprising DNA sequences that hybridize to the sequence identified in (a) above and which encode a polypeptide that is an inositol phosphorylceramide synthase; and
   c) a nucleic acid molecule comprising DNA sequences that are degenerate as a result of the genetic code to the DNA sequence defined in (a) and
   (b) and which encodes a polypeptide that is an inositol phosphorylceramide synthase.
ii) providing at least one candidate agent to be tested;
iii) forming a preparation that is a combination of (i) and (ii) above; and
iv) testing the effect of said agent on the enzyme activity of said inositol phosphoryceraminde synthase.

In a preferred method of the invention said polypeptide comprises the amino acid sequence as shown in FIG. 7B, 8B, 9B, 10B, 11B, 12B, 13B, 14B or 15B, or sequence variant thereof, wherein said variant is modified by deletion, addition or substitution of at least one amino acid residue. Preferably said polypeptide consists of the amino acid sequence shown in FIG. 7B, 8B, 9B, 10B, 11B, 12B, 13B, 14B or 15B.

According to a further aspect, there is provided a screening method for the identification of an agent that has inositol phosphoryceraminde synthase enzyme inhibitory activity comprising the steps of:
i) providing a polypeptide according to the invention;
ii) providing at least one candidate agent to be tested;
iii) forming a preparation that is a combination of (i) and (ii) above; and
iv) testing the effect of said agent on the enzyme activity of said inositol phosphoryceraminde synthase.

In a preferred method of the invention said agent comprises a ceremide moiety.

In an alternative preferred method of the invention said agent comprises a diacyl glyceride moiety.

In a further alternative preferred method of the invention said agent comprises an inositol moiety.

Preferably, said method is a cell based method, more preferable a method wherein said polypeptide is expressed by a cell according to the invention.

In a preferred method of the invention said nucleic acid molecule or said polypeptide is expressed by a cell according to the invention.

In a further preferred method of the invention said cell is a yeast cell, preferably a Saccharomyces cerevisae cell. Preferably said cell is in liquid culture medium. In an alternative preferred method of the invention said cell is grown in plate culture on solid growth medium. Preferably said agent is included in said liquid culture or solid culture medium.

The invention provides methods (also referred to herein as “screening assays”) for identifying modulators, i.e., candidate or test compounds or agents (e.g., peptides, peptidomimetics, small molecules or other drugs) which modulate inositol phosphoryceraminde synthase enzyme e.g. have a stimulatory or inhibitory effect on inositol phosphoryceraminde synthase enzyme polypeptide activity or nucleic acid expression.

The method can comprise contacting inositol phosphoryceraminde synthase or biologically active fragment thereof with a test agent and determining the ability of the agent to modulate (e.g., stimulate or inhibit) the activity of the inositol phosphoryceraminde synthase or biologically active portion thereof.

Preferably, the ability of the agent to modulate inositol phosphoryceraminde synthase activity can be determined by measuring the ability of the modulate inositol phosphoryceraminde synthase to bind modulate inositol phosphoryceraminde synthase target (e.g. by determining direct binding). In an alternative embodiment, the ability of the agent to modulate inositol phosphoryceraminde synthase activity can be determined by measuring the ability of the inositol phosphoryceraminde synthase to further modulate an inositol phosphoryceraminde synthase target, e.g, the enzymatic activity of the inositol phosphoryceraminde synthase on an appropriate substrate can be determined.

In one embodiment, the ability of the agent to modulate inositol phosphoryceraminde synthase activity can be determined by an activity assay, i.e. a modulate inositol phosphoryceraminde synthase activity assay, wherein the quantity of reaction product is determined.

In one embodiment the assay is a cell free assay. Alternatively, the assay is a cell based assay in which a cell which expresses an inositol phosphoryceraminde synthase polypeptide, or a biologically active fragment thereof.

According to a further aspect of the invention there is provided a method to determine the ability of an agent to associate with an inositol phosphorylceramide synthase polypeptide comprising the steps of:
i) providing computational means to perform a fitting operation between said agent and a polypeptide defined by the amino acid sequence in FIG. 7B, 8B, 9B, 10B, 11B, 12B, 13B, 14B or 15B; and
ii) analyzing the results of said fitting operation to quantify the association between the agent and the inositol phosphorylceramide synthase polypeptide.

The rational design of binding entities for proteins is known in the art and there are a large number of computer programs that can be utilized in the modelling of 3-dimensional protein structures to determine the binding of chemical entities to functional regions of proteins and also to determine the effects of mutation on protein structure. This may be applied to binding entities and also to the binding sites for such entities. The computational design of proteins and/or protein ligands demands various computational analyses which are necessary to determine whether a molecule is sufficiently similar to the target protein or polypeptide. Such analyses may be carried out in current software applications, such as the Molecular Similarity application of QUANTA (Molecular Simulations Inc., Waltham, Mass.) version 3.3, and as described in the accompanying User's Guide, Volume 3 pages. 134-135. The Molecular Similarity application permits comparisons between different structures, different conformations of the same structure, and different parts of the same structure. Each structure is identified by a name. One structure is identified as the target (i.e., the fixed structure); all remaining structures are working structures (i.e. moving structures). When a rigid fitting method is used, the working structure is translated and rotated to obtain an optimum fit with the target structure.

The person skilled in the art may use one of several methods to screen chemical entities or fragments for their ability to associate with a target. The screening process may begin by visual inspection of the target on the computer screen, generated from a machine-readable storage medium. Selected fragments or chemical entities may then be positioned in a variety of orientations, or docked, within that binding pocket.

Useful programs to aid the person skilled in the art in connecting the individual chemical entities or fragments include: CAVEAT (P. A. Bartlett et al, “CAVEAT: A Program to Facilitate the Structure-Derived Design of Biologically Active Molecules”. In Molecular Recognition in Chemical and Biological Problems“, Special Pub., Royal Chem. Soc., 78, pp. 182-196 (1989)). CAVEAT is available from the University of California, Berkeley, Calif. 3D Database systems such as MACCS-3D (MDL Information Systems, San Leandro, Calif.). This is reviewed in Y. C. Martin, “3D Database Searching in Drug Design”, J. Med. Chem., 35, pp. 2145-2154 (1992); and HOOK (available from Molecular Simulations, Burlington, Mass.). These citations are incorporated by reference.

Once the ligand has been optimally selected or designed, as described above, substitutions may then be made in some of its atoms or side groups in order to improve or modify its binding properties. Generally, initial substitutions are conservative, i.e., the replacement group will have approximately the same size, shape, hydrophobicity and charge as the original group. The computational analysis and design of molecules, as well as software and computer systems therefor are described in U.S. Pat. No 5,978,740 which is included herein by reference.

In a preferred method of the invention said agent comprises a ceremide moiety.

In an alternative preferred method of the invention said agent comprises a diacyl glyceride moiety.

In a further alternative preferred method of the invention said agent comprises an inositol moiety.

In a preferred method of the invention said inositol phosphorylceramide synthase polypeptide is a modified polypeptide wherein said modification is to the active site of said polypeptide wherein said modification is the addition, deletion or substitution of at least one amino acid residue such that the binding affinity and/or specificity of said agent for said active site is altered.

In a further preferred method of the invention said agent is modified to alter its binding affinity and/or specificity for said polypeptide.

In a preferred method of the invention said agent is an antagonist for said polypeptide.

In a further preferred method of the invention said polypeptide or modified polypeptide is encoded by a nucleic acid molecule as represented in FIG. 6A, 7A, 8A, 9A, 10A, 11A, 12A, 13A, 14A or 15A, or a nucleic acid molecule that hybridizes under stringent hybridization conditions to said nucleic acid molecule and encodes a polypeptide with inositol phosphorylceramide synthase activity.

According to a further aspect of the invention there is provided a method for the rational design of mutations in inositol phosphorylceramide synthase polypeptides comprising the steps of:
i) providing a 3D model of a first polypeptide as represented by the amino acid sequence in FIG. 7B, 8B, 9B, 10B, 11B, 12B, 13B, 14B or 15B;
ii) providing a 3D model of a variant polypeptide wherein said variant polypeptide is a modified sequence variant of said first polypeptide which is modified by addition, deletion or substitution of at least one amino acid residue in FIG. 7B, 8B, 9B, 10B, 11B, 12B, 13B, 14B or 15B;
iii) comparing the effect on the 3D model of said second polypeptide when compared to the 3D model of said first polypeptide; and optionally
iv) testing the effect of said modification on the enzyme activity of said second polypeptide when compared to said first polypeptide.

According to a further aspect, there is provided the use of an agent that modulates the activity an inositol phosphoryceraminde synthase enzyme in the manufacture of a medicament for the treatment of a protozoan parasitic disease.

Preferably, said protozoan parasitic disease is a kinetoplastodae disease.

Preferably, said inositol phosphoryceraminde synthase enzyme has a polypeptide sequence according to the invention.

Preferably, said protozoan parasitic disease is leishmaniasis, African trypanosomiasis or Chagas' disease.

According to a further aspect of the invention there is provided the use of an aureobasidin antibiotic in the manufacture of a medicament for the treatment of leishmaniasis.

According to a further aspect of the invention there is provided the use of an aureobasidin antibiotic in the manufacture of a medicament for the treatment of African trypanosomiasis.

According to a yet further aspect of the invention there is provided the use of an aureobasidin antibiotic in the manufacture of a medicament for the treatment of Chagas' disease.

According to a further aspect of the invention there is provided the use of a khafrefungin antibiotic in the manufacture of a medicament for the treatment of leishmaniasis.

According to a further aspect of the invention there is provided the use of a khafrefungin antibiotic in the manufacture of a medicament for the treatment of African trypanosomiasis.

According to a yet further aspect of the invention there is provided the use of a khafrefungin antibiotic in the manufacture of a medicament for the treatment of Chagas' disease.

According to a further aspect of the invention there is provided the use of a rustmicin antibiotic in the manufacture of a medicament for the treatment of leishmaniasis.

According to a further aspect of the invention there is provided the use of a rustmicin antibiotic in the manufacture of a medicament for the treatment of African trypanosomiasis.

According to a yet further aspect of the invention there is provided the use of a rustmicin antibiotic in the manufacture of a medicament for the treatment of Chagas' disease.

According to a further aspect of the invention there is provided a method to treat leishmaniasis comprising administering an effective amount of at least one antibiotic selected from the group consisting of: aureobasidin, khafrefungin or rustmicin to a subject in need of treatment.

According to a further aspect of the invention there is provided a method to treat African trypanosomiasis comprising administering an effective amount of at least one antibiotic selected from the group consisting of aureobasidin, khafrefungin or rustmicin to a subject in need of treatment.

According to a further aspect of the invention there is provided a method to treat Chagas' disease comprising administering an effective amount of at least one antibiotic selected from the group consisting of: aureobasidin, khafrefungin or rustmicin to a subject in need of treatment.

In a preferred method of the invention said subject is a human.

In a further aspect the invention provides the use of an agent identified by a method according to the invention that modulates the activity an inositol phosphoryceraminde synthase enzyme in the manufacture of a medicament for the treatment of a protozoan parasitic disease.

When administered, the medicaments of the present invention are administered in pharmaceutically acceptable preparations. Such preparations may routinely contain pharmaceutically acceptable concentrations of salt, buffering agents, preservatives, compatible carriers, supplementary immune potentiating agents such as adjuvants and optionally other therapeutic agents.

The medicaments of the invention can be administered by any conventional route, including injection or by gradual infusion over time. The administration may, for example, be oral, intravenous, intraperitoneal, intramuscular, intracavity, subcutaneous, or transdermal.

The medicaments of the invention are administered in effective amounts. An “effective amount” is that amount of a medicament that alone, or together with further doses, produces the desired response. In the case of treating a particular disease such as leishmaniasis or trypanosomiasis, the desired response is inhibiting the progression of the disease. This may involve only slowing the progression of the disease temporarily, although more preferably, it involves halting the progression of the disease permanently. This can be monitored by routine methods.

Such amounts will depend, of course, on a number of criteria, for example, the severity of the condition, the individual patient parameters including age, physical condition, size and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reasons.

The medicaments used in the foregoing methods preferably are sterile and contain an effective amount of agent for producing the desired response in a unit of weight or volume suitable for administration to a patient. The doses of agent administered to a subject can be chosen in accordance with different parameters, in particular in accordance with the mode of administration used and the state of the subject. Other factors include the desired period of treatment. In the event that a response in a subject is insufficient at the initial doses applied, higher doses (or effectively higher doses by a different, more localized delivery route) may be employed to the extent that patient tolerance permits.

When administered, medicaments of the invention are applied in pharmaceutically-acceptable amounts and in pharmaceutically-acceptable compositions. The term “pharmaceutically acceptable” means a non-toxic material that does not interfere with the effectiveness of the biological activity of the active ingredients. Such preparations may routinely contain salts, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

Medicaments may be combined, if desired, with a pharmaceutically-acceptable carrier. The term “pharmaceutically-acceptable carrier” as used herein means one or more compatible solid or liquid fillers, diluents or encapsulating substances which are suitable for administration into a human. The term “carrier” denotes an organic or inorganic ingredient, natural or synthetic, with which the active ingredient is combined to facilitate the application.

The medicaments may contain suitable buffering agents, including: acetic acid in a salt; citric acid in a salt; boric acid in a salt; and phosphoric acid in a salt. The medicaments also may contain, optionally, suitable preservatives, such as: benzalkonium chloride; chlorobutanol; or parabens.

The medicaments may conveniently be presented in unit dosage form and may be prepared by any of the methods well-known in the art of pharmacy. All methods include the step of bringing the active agent into association with a carrier which constitutes one or more accessory ingredients. In general, the medicaments are prepared by uniformly and intimately bringing the active compound into association with a liquid carrier, a finely divided solid carrier, or both, and then, if necessary, shaping the product. Medicaments suitable for oral administration may be presented as discrete units, such as capsules, tablets, lozenges, each containing a predetermined amount of the active compound. Other compositions include suspensions in aqueous liquids or non-aqueous liquids such as syrup, elixir or an emulsion.

Medicaments suitable for parenteral administration conveniently comprise a sterile aqueous or non-aqueous preparation of agent, which is preferably isotonic with the blood of the recipient. This preparation may be formulated according to known methods using suitable dispersing or wetting agents and suspending agents. The sterile injectable preparation also may be a sterile injectable solution or suspension in a non-toxic parenterally-acceptable diluent or solvent, for example, as a solution in 1, 3-butane diol. Among the acceptable vehicles and solvents that may be employed are water, Ringer's solution, and isotonic sodium chloride solution.

In addition, sterile, fixed oils are conventionally employed as a solvent or suspending medium. For this purpose any bland fixed oil may be employed including synthetic mono-or di-glycerides. In addition, fatty acids such as oleic acid may be used in the preparation of injectables. Carrier formulation suitable for oral, subcutaneous, intravenous, intramuscular, etc. administrations can be found in Remington's Pharmaceutical Sciences, Mack Publishing Co., Easton, Pa.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

An embodiment of the invention will now be described by example only and with reference to the following figures:

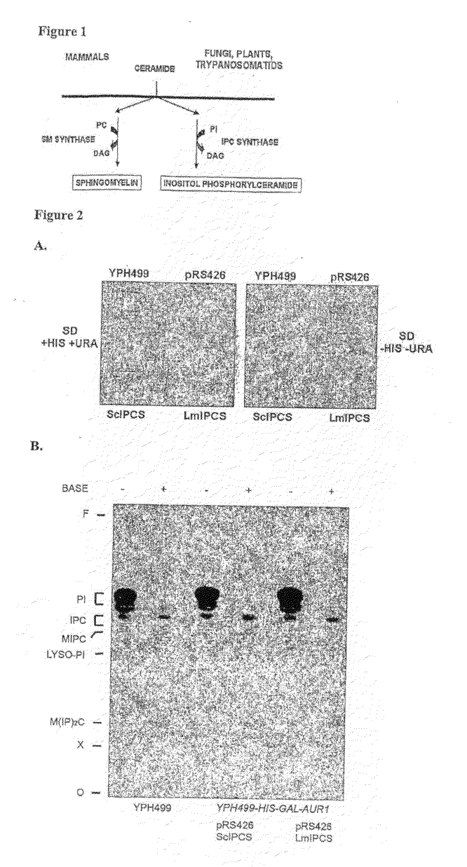

FIG. 1: Schematic illustrating the dichotomy of complex sphingolipid biosynthesis: mammals (and other animals) producing sphingomyelin (SM) via SM synthase; and fungi, plants and kinetoplastids synthesizing inositol phosphorylceramide (IPC) utilizing IPC synthase.

FIG. 2: A. Rescue of YPH499-HIS-GAL-AUR1 by complementation with either the yeast IPC synthase or a predicted Leishmania orthologue identified using a bioinformatic approach. YPH499—wild type; pRS426—YPH499-HIS-GAL-AUR1 pRS426MET25; ScIPCS—YPH499-HIS-GAL-AUR1 pRS426 ScIPCS; LmIPCS—YPH499-HIS-GAL-AUR1 pRS426 LmIPCS. SD—synthetic minimal media supplemented (or not) with histidine (HIS) and uracil (URA) for selection of the pRS426MET25 plasmid. B. Complementation with either the yeast or protozoan open reading frame facilitates synthesis of a base resistant sphingolipid profile identical to that previously characterized in wild type yeast (31). Material extracted from wild type YPH499 and the auxotrophic mutant YPH499-HIS-GAL-AUR1 complemented with pRS426 ScIPCS or pRS426 ScIPCS. O—origin; F—front; X—unknown; PI—phosphatidylinositol; IPC—inositol phosphorylceramide; MIPC—mannose inositol phosphorylceramide; M(IP)₂C—mannose (inositol phosphorus)₂ ceramide.

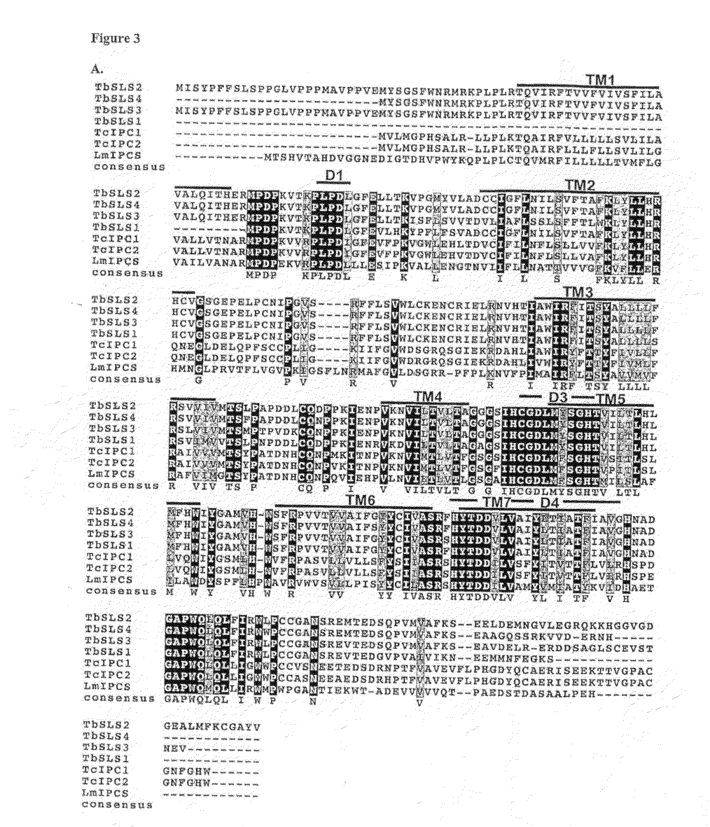

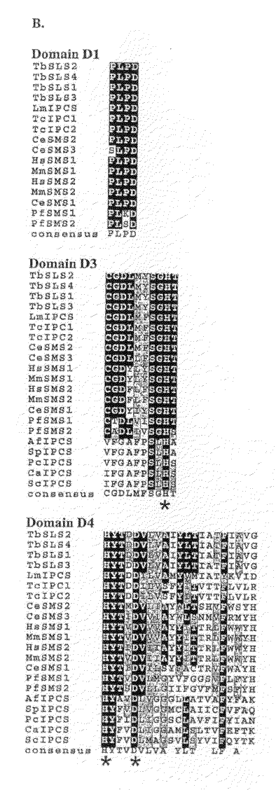

FIG. 3: A. The kinetoplastid sphingolipid synthases are highly conserved. CLUSTALW alignment. TM—predicted LmIPCS transmembranes domains; D—conserved SM synthase domains. Shaded black—100% identical; shaded grey—100% similarity. B.

The kinetoplastid sphingolipid synthases share a high level of similarity with the SM synthases with respect to 3 domains. Edited CLUSTALW alignments of the conserved SM synthase domains D1, D3 and D4. Equivalent to LmIPCS amino acids 67-70, 212-221 and 264-284 respectively. Shaded black—50% identical; shaded grey—50% similarity. *—conserved histidine and aspartate residues that form the catalytic triad. C. The kinetoplastid proteins define a new class of sphingolipid synthases. An evolutionary tree constructed using maximum parasimony of sequence equivalent to LmIPCS amino acids 77-313, a region conserved between the Metazoa, Fungi, Apicomplexa and Kinetoplastidae and including the domains within which the amino acids forming the catalytic triad are present. The sequence of the human lipid phosphate phosphatase 1 (LPP1) was the designated outgroup. Bootstrap score >60 indicated

T. brucei SLS1-4 accession numbers: Tb09.211.1020, Tb09.211.1000, Tb09.211.1030, Tb09.211.1010; T. cruzi IPCS1&2: Tc00.1047053506885.124, Tc00.1047053510729.290; L. major IPCS: LmjF35.4990; Aspergillus fumigatus IPCS: AAD22750; Candida albicans IPCS: AAB67233; Pneumocystis carinii IPCS: CAH17867; Saccharomyces cerevisiae IPCS: NP_{—}012922; Schizosaccharomyces pombe IPCS: Q10142; Plasmodium falciparum SMS 1&2: MAL6P1.178, MAL6P1.177; Caenorhabditis elegans SMS1-3: Q9U3D4, AAA82341, AAK84597; Homo sapiens SMS1&2: AB154421, Q8NHU3; Mus musculus SMS1&2: Q8VCQ6, Q9D4B1; Homo sapiens LPP1 (outgroup): 014494.

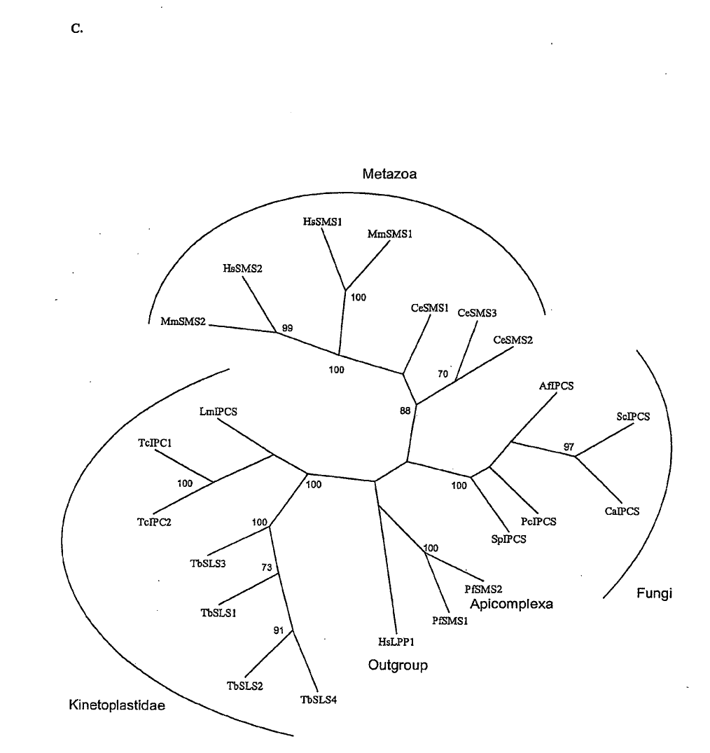

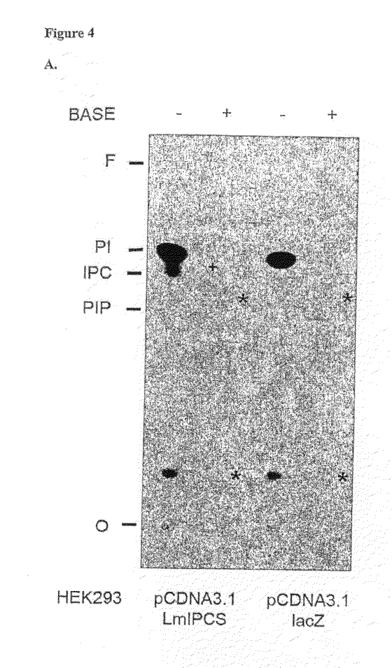

FIG. 4 A. LmIPCS constituted IPC synthase activity in mammalian HEK293 cells. HEK293 transiently expressing LmIPCS from pcDNA3.1 synthesized a base resistant, inositol-labelled lipid species (+) that migrated with inositol phosphorylceramide. This lipid was absent in cells treated identically but which expressed lacZ. PI—phosphatidylinositol; IPC—inositol phosphorylceramide. Both lines demonstrated base resistant inositol-labelled lipid species without equivalents in untreated cells (*). These were assumed to be breakdown products of base sensitive inositol lipid species. O—origin; F—front; PI—phosphatidylinositol; IPC—inositol phosphorylceramide; PIP—phosphatidylinositol phosphate. B.LmIPCS localizes to the Golgi apparatus in mammalian HEK293 cells. i) DAPI stained ii) LmIPCS; ; iii) Human ARF1-GFP; iv) merge of i-iv; v) DIC. C. The catalytic triad of amino acids is orientated towards the Golgi lumen. Predicted membrane topology of LmIPCS.

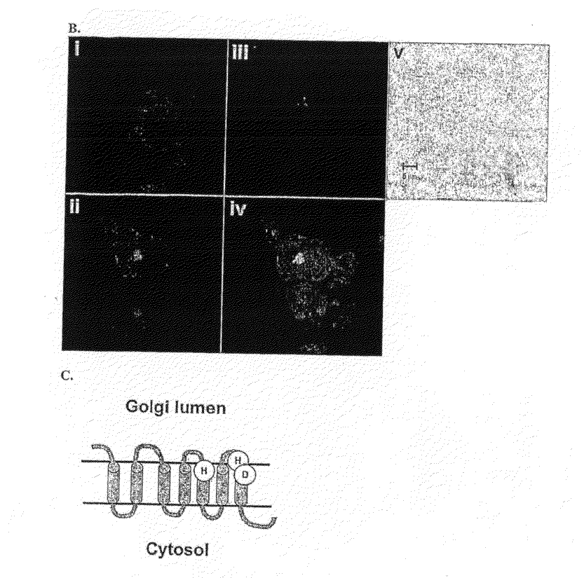

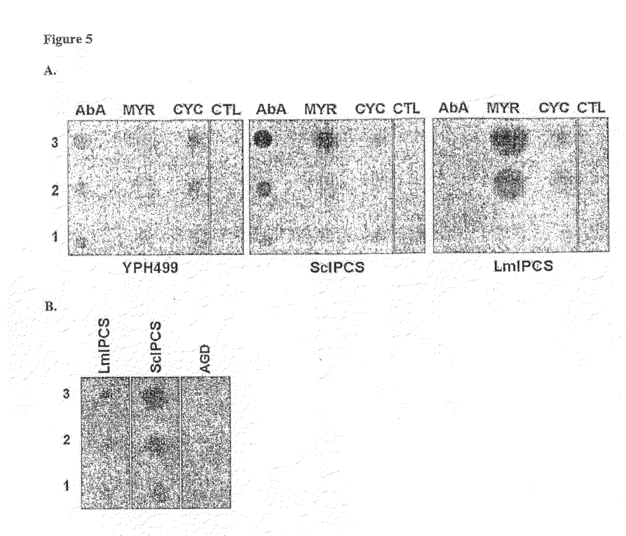

FIG. 5 LmIPCS is specifically susceptible to the fungal inhibitor Aureobasidin A. Agar diffusion assay. A. Volumes of 1, 2 and 3 μl of 25 μM Aureobasidin A (AbA), 1 mM myriocin (MYR), 25 μM cycloheximide (CYC) and DMSO as a control (CTL) spotted onto yeast plates prepared as described in Experimental Procedures. B. Volumes of 1, 2 and 3 μl of 100 μM Aureobasidin A spotted onto yeast plates prepared as described in Experimental Procedures.

YPH499—wild type yeast; ScIPCS—YPH499-HIS-GAL-AUR1 pRS426 ScIPCS; LmIPCS—YPH499-HIS-GAL-AUR1 pRS426 LmIPCS; AGD—yeast sphingolipid bypass mutant, negative control.

FIG. 6A is the nucleic acid sequence of Leishmania major inositol phosphorylceramide synthase; FIG. 6B is the amino acid sequence of Leishmania major inositol phosphorylceramide synthase;

FIG. 7A is the nucleic acid sequence of TbIPCS 1 Trypanosoma brucei inositol phosphorylceramide synthase; FIG. 7B is the amino acid sequence of TbIPCS 1 Trypanosoma brucei inositol phosphorylceramide synthase;

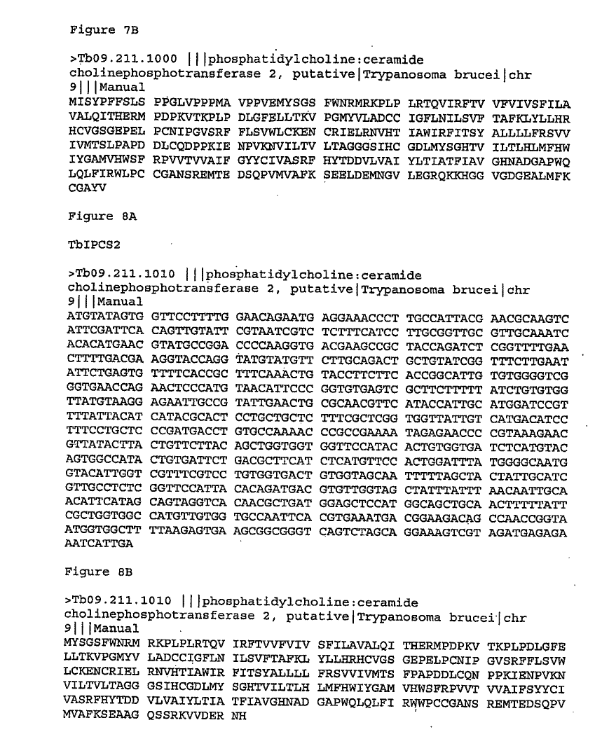

FIG. 8A is the nucleic acid sequence of TbIPCS 2 Trypanosoma brucei inositol phosphorylceramide synthase; FIG. 8B is the amino acid sequence of TbIPCS 2 Trypanosoma brucei inositol phosphorylceramide synthase;

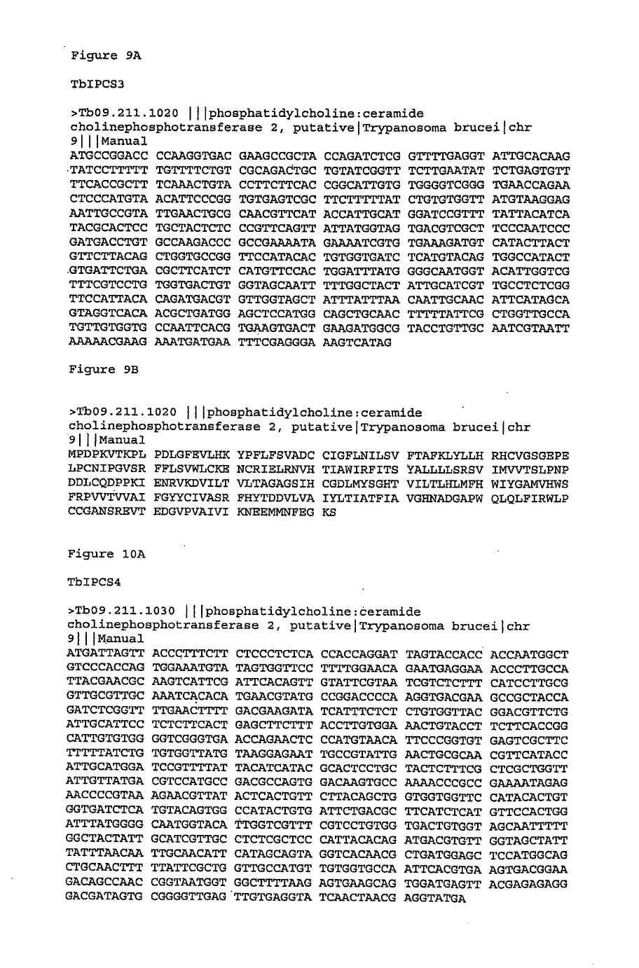

FIG. 9A is the nucleic acid sequence of TbIPCS 3 Trypanosoma brucei inositol phosphorylceramide synthase; FIG. 9B is the amino acid sequence of TbIPCS 3 Trypanosoma brucei inositol phosphorylceramide synthase;

FIG. 10A is the nucleic acid sequence of TbIPCS 4 Trypanosoma brucei inositol phosphorylceramide synthase; FIG. 8B is the amino acid sequence of TbIPCS 4 Trypanosoma brucei inositol phosphorylceramide synthase;

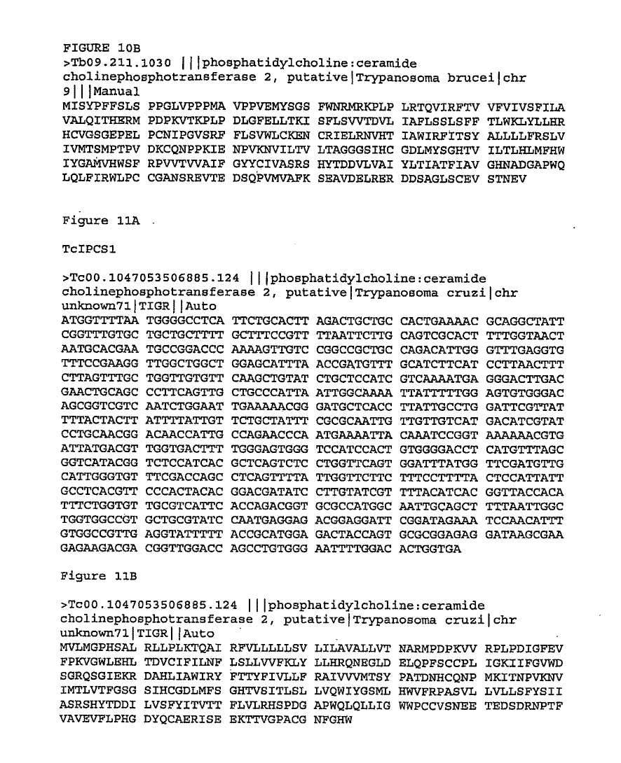

FIG. 11A is the nucleic acid sequence of TcIPCS 1 Trypanosoma cruzi inositol phosphorylceramide synthase; FIG. 8B is the amino acid sequence of TcIPCS 1 Trypanosoma cruzi inositol phosphorylceramide synthase;

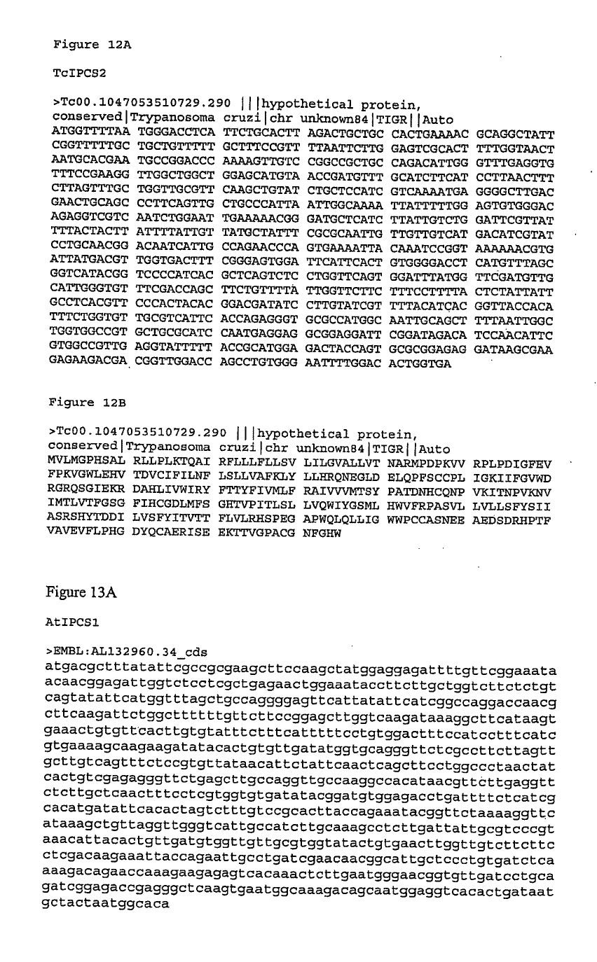

FIG. 12A is the nucleic acid sequence of TcIPCS 2 Trypanosoma cruzi inositol phosphorylceramide synthase; FIG. 12B is the amino acid sequence of TcIPCS 2 Trypanosoma cruzi inositol phosphorylceramide synthase;

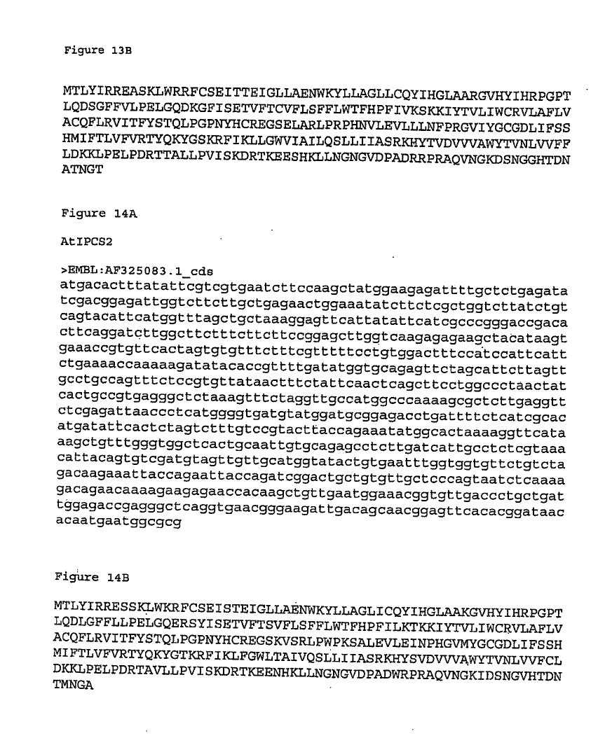

FIG. 13A is the nucleic acid sequence of AtIPCS 1 Arabidopsis thaliana inositol phosphorylceramide synthase; FIG. 8B is the amino acid sequence of AtIPCS 1 Arabidopsis thaliana inositol phosphorylceramide synthase; and

FIG. 14A is the nucleic acid sequence of AtIPCS 2 Arabidopsis thaliana inositol phosphorylceramide synthase; FIG. 14B is the amino acid sequence of AtIPCS 2 Arabidopsis thaliana inositol phosphorylceramide synthase;

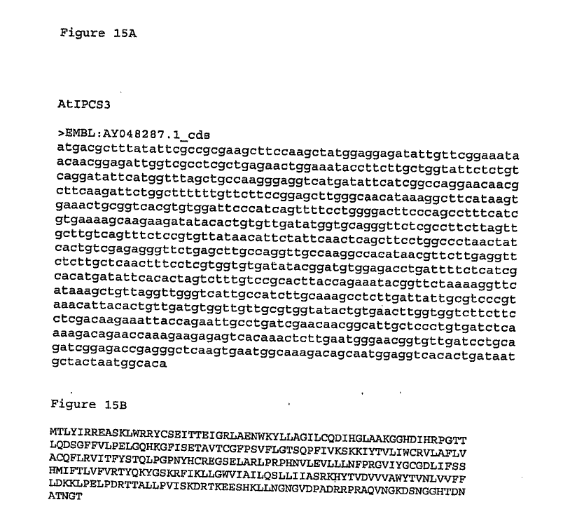

FIG. 15A is the nucleic acid sequence of AtIPCS 3 Arabidopsis thaliana inositol phosphorylceramide synthase; FIG. 15B is the amino acid sequence of AtIPCS 3 Arabidopsis thaliana inositol phosphorylceramide synthase; and

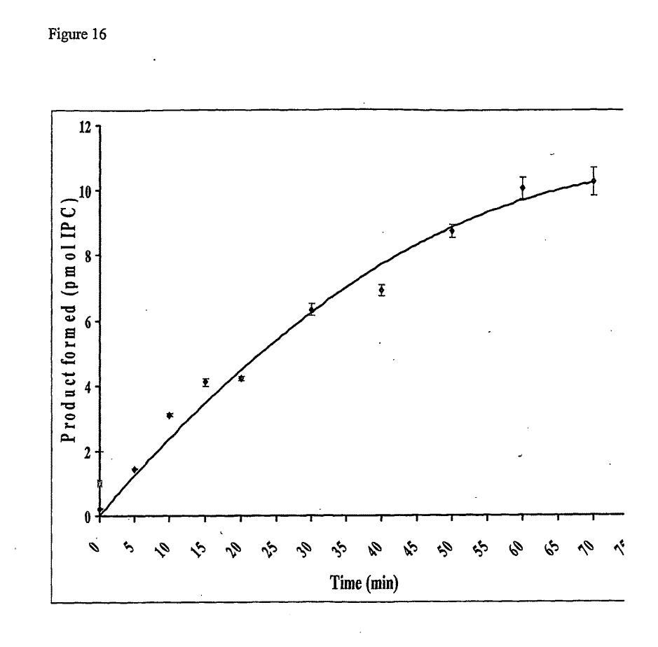

FIG. 16 is an activity curve: CHAPS-Washed LimIPCS Activity vs. Time (Each reaction contained 1 μg protein, 1 nMol PI, 50 pMol NBD-C6-Ceramide and 0.2 μMol CHAPS).

### MATERIALS AND METHODS

Selection and Cloning of a Candidate IPC Synthase Gene

Candidate genes were identified in a Motif Search of the genome sequence databases of L. major, T. brucei and T. cruzi using the sequence motif H-[YFWH]-X₂-D-[VLI]-X₂-[GA]-X₃-[GSTA] (Sanger GeneDB). Candidates were then further selected using the additional criteria described in the Results.

The Leishmania candidate, CDS—LmjF35.4990, was amplified from genomic DNA (8) using Pfu polymerase (Stratagene) and the primer pairs: 5′LmIPCSBamHI (5′cgc gga tcc ATG ACG AGT CAC GTG ACA GC) and 3′LmIPCSHindIII w/* (ccc aag ctt TTA GTG CTC AGG CAA AGC CGC CG); 5′LmIPCSBamHI and 3′LmIPCSHindIII (ccc aag ctt GTG CTC AGG CAA AGC CGC CG). The products were cloned into the yeast and mammalian expression vectors pRS426MET25 and pcDNA3.1/Myc-HisA (Invitrogen), creating pRS426 LmIPCS and pcDNA3.1A LmIPCSMyc-His respectively. The S. cerevisiae AUR1 gene was similarly amplified from genomic DNA (Invitrogen) using the primer pair AURFXbaI (cat aga tct aga ATG GCA AAC CCT TT TTC GAG ATG G) and 3′ScIPCSHindIII w/* (ccc aag ctt TTA AGC CCT CTT TAC ACC TAG TGA CG), and the product cloned into pRS426MET25 to create pRS426 ScIPCS. Cloning sites are shown in lower case, with Leishmania and S. cerevisiae sequence in upper case.

Construction of YPH499-HIS-GAL-AUR1 auxotrophic S. cerevisiae strain—YPH499-HIS-GAL-AUR1 S. cerevisiae strain was constructed in YPH499 [Mat a; ura3-52; lys2-801amber; ade2-101ochre; trp1-63 ; his3-200; leu2-1] (Stratagene) by bringing the expression of the yeast AUR1 gene under the control of the stringently regulated GAL1 promoter that is repressed in the presence of glucose (12). The AUR1 promoter in the yeast genome was exchanged by a selection marker/promoter HIS/GAL1-cassette using previously described methodology (13). The primer sequences for amplification of the HIS-GAL cassette were chosen as follows: (a) sequence for integration upstream of the coding region (nucleotides −200 to −150)—AuriHISGalS 5′GGT AGT TGG TTA GTC CGA TCG CTC ACT TTT GGT TGT TGT TAA GTA CTT CAG GGC GAA TTG GAG CTC CAC3′; (b) sequence for integration at the initiation codon (nucleotides +1 to +50)—AuriHISGalAS 5′CAG TTT GGA GGT CTC TCT GAT AGA AAC CAT CTC GAA AAA GGG TTT GCC ATG GGG ATC CAC TAG TTC TAG3′. The numbers indicate the nucleotide positions in the S. cerevisiae DNA sequence, with the adenosine of the ATG initiation codon being defined as position +1. The 19 by sequences at the 3′ ends of these oligonucleotides that are homologous to the sequences of the vector pGAL/HIS3 and serve as a template for amplification of the GAL1/HIS3-cassette are underlined. Transformation into the hapolid YPH499 strain, selection on minimal medium lacking histidine but containing galactose and confirmation of the insertion of the HIS-GAL fragment were performed as previously (14). YPH499-HIS-GAL-AUR1 was maintained in SGR medium (4% galactose, 2% raffinose, 0.17% Bacto yeast nitrogen base, 0.5% ammonium sulphate) with galactose/raffinose rather than non-permissive dextrose as the carbohydrate source. For rapid cultivation of the mutant YPGR medium (4% galactose, 2% raffinose, 1% yeast extract, 2% peptone) was routinely used.

Identification of Leishmania IPC Synthase

The YPH499-HIS-GAL-AUR1 S. cerevisiae strain was transformed with pRS426 ScIPCS or pRS426 LmIPCS and functionally complemented transformants selected on non-permissive SD medium (0.17% Bacto yeast nitrogen base, 0.5% ammonium sulphate and 2% dextrose) containing the nutritional supplements necessary to allow selection of transformants.

Sequence analyses—Kinetoplast orthologues of LmIPCS (CDS—LmjF35.4990) were found by BLAST search of the T. brucei and T. cruzi databases (Sanger GeneDB) using WU-BLASTp (Gish, W. (1996-2001) http://blast.wustl.edu). Sequence alignments were made using CLUSTALW (15). Phylogenetic analyses were performed on the edited alignments using maximum parsimony (Protpars, Felsenstein, J. 1993. PHYLIP (Phylogeny Inference Package version 3.5c; distributed by the author, Department of Genetics, University of Washington, Seattle, USA). Topology predictions were performing using the PHD package (16).

Expression of Leishmania IPC synthase in a mammalian cell line—Human ARF1 was amplified from lymph node cDNA (Clontech) using the primers hARF1F (5′CCT GTC CAC AAG CAT GGG GAA CATS′ and hARF1R, 5′CCT TCT GGT TCC GGA GCT GAT TGG3′. The fragment was cloned into pcDNA3.1/CT-GFP-TOPO (Invitrogen) to produce the construct pcDNA3.1 hARF1GFP.

The cell line HEK293 was grown in Dulbecco's modified eagle medium (DMEM), containing 10% FCS, 100 U/ml penicillin and 100 U/ml streptomycin at 37° C. in a humidified atmosphere with 5% CO₂.

For transient transfection, cells were seeded in 6-well plates+/−glass coverslips at a cell density of 2×10⁵ cells per well, and allowed to grow for 24 hours. Cells were then transfected with pcDNA3.1A LmIPCSMyc-His, pcDNA3.1 hARF1GFP or pcDNA3.1A vector alone, using Fugene 6 Transfection Reagent (Roche). For each well, 3 μl of Fugene reagent was combined with 2 μg of plasmid DNA. For co-transfections, 6 μl of Fugene was used per well, with 2 μg of each construct.

Immunofluorescence

36 hours post-transfection HEK293cells were fixed with 4% paraformaldehyde (w/v) for 45 minutes at RT. Expressed LmIPCS was detected by indirect immunofluorescence. Cells were washed in PBS, permeabilized in 0.5% Triton X-100/PBS (v/v) for 10 mins and then blocked in 10% FCS/PBS (v/v) for 1 hour. Samples were incubated with rabbit polyclonal anti-myc (Abeam, 1:200 dilution) in blocking solution for 1 hour. After washing in PBS, cells were incubated in Alexa Fluor 633 goat anti-rabbit IgG (Invitrogen, 1:250) in blocking solution for 1 hour. After washing in PBS, coverslips were mounted on slides using Vectashield with DAPI (Vector Laboratories, UK). Samples were visualized by confocal microscopy using a Zeiss LSM 510 meta on an Axiovert 200M, with a Plan-Apochromat 63x/1.4 Oil DIC I objective lens. Images were acquired using LSM 510 version 3.2 software (Zeiss).

Metabolic Labelling and Analyses

HEK293 cells, 36 hours post-transfection, were labelled for 16 hours in DMEM (ICN) supplemented with 10% foetal calf serum (Gibco BRL) and 20 μCi/ml of myo-[³H] inositol (102 Ci/mmol) (Amersham). Lipids were extracted and analyzed as previously described (17,18).

Logarithmic phase yeast were harvested and incubated at 10 OD⁶⁰⁰/ml in 0.25 ml inositol-free SD medium at 30° C. for 20 minutes. 10 μCi [³H]inositol were added and the cells incubated for 40 minutes. 0.75 ml of fresh medium was added and the incubation continued for 80 minutes. Cells were harvested by centrifugation and washed twice with 1 M sorbitol. Chloroform/methanol (0.4 ml; 1:1 v/v) was added and cells were disintegrated with glass beads. The pellet was re-extracted several times with chloroform/methanol/water (10:10:3, per vol.), the combined supernatants dried and the lipids prepared and analyzed as previously described (17,18).

Agar diffusion assay—Wild type YPH499 yeast and the transgenic strain YPH499-HIS-GAL-AUR1 complemented with ScIPCS or LmIPCS were assayed for susceptibility to Aureobasidin A (Takara), myriocin (Sigma) and cycloheximide (Sigma) as previously described (19). Briefly, 2.4×10⁷ logarithmically dividing cells were embedded in 15 ml of YPD agararose (2% dextrose, 1% yeast extract, 2% peptone, 0.8% agarose) on 100 mm² square Petri dishes (Sarstedt). Inhibitors were applied in DMSO at the concentrations described below and the dishes incubated at 30° C.

### Examples

Functional Cloning of Leishmania IPC Synthase

Candidate genes encoding the protozoan enzyme were selected using a bioinformatic approach based on that used in the identification of the metazoan enzyme sphingomyelin (SM) synthase (20). Briefly, a conserved motif shared by the lipid phosphate phosphatase (LPP) family and both animal and fungal sphingolipid synthases, H-[YFWH]-X₂-D-[VLI]-X₂-[GA]-X₃-[GSTA], was used to screen the genome sequence databases of the kinetoplastids (Sanger GeneDB). Of the 8 candidates identified, one was of unknown function, had orthologues in both Trypanosoma and Leishmania spp., had transmembrane domains consistent with a Golgi localized IPC synthase and had no orthologue in mammalian cells (which do not express IPC activity). The gene encoding this putative protozoan IPC synthase is present as a single copy in Leishmania. In contrast the Trypanosomes possess multiple non-identical loci, 2 in T. cruzi, and 4 in T. brucei. The Leishmania candidate was amplified and cloned into a URA3 selectable yeast expression vector to create pRS426 LmIPCS. This construct restored the growth of YPH499-HIS-GAL-AUR1 (see Experimental Procedures) in non-permissive SD media, as did the ectopic expression of S. cerevisiae AUR1p (ScIPCS) (FIG. 2A). IPC synthase is an essential activity in fungi (21), therefore the non-complemented mutant line is unable to propagate in the absence of galactose and AUR1p expression. The wild type (YPH499) cannot grow without uracil.

Biochemical analysis of these complemented yeast cell lines validated LmIPCS as being fully functional in the auxotrophic S. cerevisiae mutant. The YPH499-HIS-GAL-AUR1 rescued using either pRS426 ScIPCS or pRS426 LmIPCS and labelled with [³H]-inositol demonstrated, following fractionation by thin-layer chromatography, a base-resistant sphingolipid profile identical to that observed in the wild type (FIG. 2B). Taken together these data establish LmIPCS as a functional orthologue of the S. cerevisiae enzyme, ScIPCS.

Leishmania IPC Synthase Defines a New Class of Sphingolipid Synthase

Orthologues of the LmIPC synthase were, as described in Experimental Procedures, identified in the related kinetoplastids T. cruzi and T. brucei. This demonstrated the IPC synthase to be a ubiquitous feature of this important group of flagellated parasites. Like Leishmania, T. cruzi synthesizes IPC as its predominant complex sphingolipid and this parasite is known to possess IPC synthase activity (22). Therefore the 2 closely related T. cruzi predicted proteins, sharing 52-53% sequence identity and 69-70% similarity with the Leishmania orthologue, were designated TcIPCS 1 and TcIPCS2. There are no published data indicating whether T. brucei produce IPC or SM. Given this uncertainty the 4 predicted orthologues were designated as T. brucei sphingolipid synthase (TbSLS) 1-4. The 4 TbSLS predicted proteins share 43-44% identity and 61-63% similarity with LmIPCS. The high level of conservation within these kinetoplastid species is illustrated in FIG. 3A, together with the 7 transmembrane domains identified during the topology prediction of LmIPCS and those regions conserved with respect to the animal SM synthases (20). SM synthase motifs D3 (C-D-G-X₃-S-G-H-T) and D4 (H-Y-T-X-D-V-X₃-Y-X₆-F-X₂-Y-H) are similar to the C2 and C3 motifs in LPP (20). These regions contain the histidine and aspartate residues (underlined) making up the catalytic triad that mediates nucleophilic attack on lipid phosphate ester bonds (20,23). Huitema et al (2004) predict that the D1 (P-L-P-D) and D2 (R-R-X₈-Y-X₂-R-X₆-T) motifs are entirely unique to the SM synthases. The fungal IPC synthase also encodes domains (3 and 4) with similarity to LPP C2 and C3 and which maintain the residues forming the catalytic triad, mutagenesis of these has been shown to inactivate the IPC synthase activity (24).

Analyzing the conserved domains D1, D3 and D4 in isolation (FIG. 3B) it is clear that the kinetoplastid sphingolipid synthases possess the histidine and aspartate residues forming the catalytic triad. However, it is also evident that they demonstrate a higher degree of similarity in these regions with the animal SM synthases than with the fungal IPC synthases. This is clearly illustrated in the complete identity of D1, a domain with no equivalent in the fungal enzyme. Notably, both mammal (20) and Leishmania (25) sphingolipid synthases utilize ceramide (rather than phytoceramide as in fungi), the conserved D1 domain may function in the preferential binding of this substrate. However, perhaps significantly, the protozoan proteins do not possess a motif with similarity to SM synthase D2.

These data indicate that the protozoan enzymes are not closely related to fungal IPC synthase, despite exhibiting an equivalent function at least in the case of Leishmania. Further, phylogenetic analysis of both SM and IPC synthase sequences using maximum parsimony demonstrated that the kinetoplastid proteins form a distinct Glade and as such define a new class of sphingolipid synthases (FIG. 3C). This analysis failed to place the putative SM synthase of the malaria parasite, Plasmodium falciparum (PfSMS) (20), raising a question over its classification and evolutionary origin.

Functional Analyses of Leishmania IPC Synthase in a Mammalian Cell Line

As described above, mammalian cells do not possess either IPC synthase or IPC (FIG. 1). In order to establish whether the protozoan gene isolated was sufficient to constitute enzyme activity, epitope-tagged LmIPCS was ectopically expressed in HEK293 cells and the cells subsequently labelled with [³H]-inositol to assay for the synthesis of IPC. Fractionation of labelled lipids by thin-layer chromatography demonstrates the presence of a base-resistant inositol-containing lipid migrating with IPC (FIG. 3A). The negative control, HEK293 cells transfected with lacZ, do not synthesize this lipid species. This result demonstrated that the gene identified encodes for a protein that can constitute IPC synthase activity in a null system.

AUR1p has been localized to the Golgi apparatus in S. cerevisiae (24), whilst in mammalian cells the 2 distinct isoforms of SM synthase have been localized to the Golgi and the plasma membrane (20). Immuno-localization of epitope-tagged LmIPCS in HEK293 cells demonstrated that the protozoan enzyme is concentrated within the Golgi apparatus as identified by the association of a GFP-tagged marker, ARF 1 (FIG. 4B), however minor quantities of the protein also localize to the plasma membrane when the expression levels are high as in FIG. 4B. Topology prediction using the PHD package indicates that the residues predicted to form the LmIPCS catalytic triad are orientated towards the Golgi lumen (or the cell surface) in a conformation identical to that predicted for both fungal IPC and animal SM synthases (20,24) (FIG. 4C). These observations suggest that, as in animals and fungi, kinetoplastid sphingolipid synthesis takes place in the lumen of the Golgi.

Specific Inhibition of Leishmania IPC Synthase

The expression of functional protozoan orthologues in the mutant yeast system described (FIG. 2) provided an ex vivo system in which to screen the existing fungal IPCS inhibitors against the functional Leishmania orthologue. In this study, the diffusion assay system previously described, with the sphingolipid bypass mutant (AGD) as a negative control, was utilized (6,19). The conditional AUR1 mutant rescued with ScIPCS was, like the wild type control, sensitive to cycloheximide (an inhibitor of protein translation), myriocin (an inhibitor of SPT which mediates the first step in sphingolipid biosynthesis) and Aureobasidin A (an inhibitor of fungal IPC synthase). The mutant rescued with LmIPCS was sensitive to cycloheximide and myriocin, however it was resistant to Aureobasidin A at 25 μM (FIG. 5A). However, by increasing the concentration of the IPC synthase inhibitor 4 fold (to 100 μM) sensitivity of LmIPCS to the drug was evident (FIG. 5B). Strain AGD grows without a functional SPT and as such IPC synthase is redundant, therefore it is resistant to anti-fungals targeting either of these enzymes provided that these agents have a specific mode of action. The lack of an inhibitory effect of Aureobasidin A at 100 μM on the growth of AGD, together with its action against the rescued mutant, demonstrates that the drug specifically inhibited LmIPCS as well as ScIPCS (19). The LmIPCS rescued mutant showed increased sensitivity to myriocin (an inhibitor of SPT) when compared with either the wild-type or the mutant rescues with ScIPCS (FIG. 5A). The reason for this is unclear, however it correlates with the generally fragile nature of the cells complemented with the protozoan orthologue as evidenced by a reduced rate of growth (data not shown).

Aureobasidin A (at 20 μM) also exhibited an effect against insect stage Leishmania in culture. However, in this system the inhibitor was non-specific as evidenced by an equivalent activity against parasites without an active sphingolipid biosynthetic pathway (8) in which LmIPCS is redundant (data not shown).

Sphingolipids are essential membrane components of eukaryotic cells. In mammalian systems the major complex sphingolipid is sphingomyelin, whilst in the kinetoplastids, including Leishmania spp., it is inositol phosphorylceramide (IPC). A similar situation exists in fungi where IPC is synthesized utilizing the enzyme IPC synthase. The fungal IPC synthase, encoded by the AUR1 gene, has been demonstrated to be essential for viability (26). No orthologue of the AUR1 gene could be identified the complete kinetoplastid genome databases, however in the study reported here we have isolated the gene encoding the Leishmania IPC synthase (LmIPCS) utilizing a combination of bioinformatics and functional genetics. The protein encoded by this gene exhibits only limited homology to fungal IPC synthase, however orthologues exist in both the African and American Trypanosomes, the causative agents of African sleeping sickness and Chagas” Disease respectively. Notably, the kinetoplastid sphingolipid synthases demonstrate significant homology to 3 of the 4 conserved domains of the recently identified animal sphingomyelin (SM) synthases (20). This indicates that, despite possessing an equivalent function, the protozoan enzymes are not closely related to the fungal IPC synthases, rather they possesses more similarity, at least at a primary sequence level, with the animal SM synthases. Indeed, phylogenetic analyses demonstrated that the kinetoplastid proteins define a new class of sphingolipid synthases clustering with neither the fungal or animal sequences (FIG. 3C). The significance of this in evolutionary terms is unclear, and the identification of a plant IPC synthase will facilitate more informative analyses.

In addition to demonstrating functionality in a yeast mutant strain, LmIPCS was also able to constitute enzyme active in cultured mammalian cells, a system that is null for IPC and IPCS. This represents the first demonstration of IPC synthase activity in an axenic system and indicates that LmIPC alone is sufficient for enzyme function. This functionality is largely confined to the Golgi apparatus as indicated by subcellular localisation of LmIPCS in the mammalian system, a situation similar to that for sphingolipid synthesis in both fungi and animals (20,24). The association of LmIPCS with membranes is predicted to be mediated by seven transmembrane helices, with the putative active site including the catalytic triad of histidine and aspartate residues orientated towards the lumen of the Golgi as in fungal and mammalian sphingolipid synthases (20,24). This strongly implies that the protozoan, animal and fungal sphingolipid synthases possess a conserved mechanism of action similar to that described for LPPs (23) where, in the case of the yeast and kinetoplastids, IPC synthase catalyses the transfer of a inositol phosphate group from phosphatidylinositol to the 1-hydroxy group of ceramide or phytoceramide releasing diacyl glycerol as a by-product. This is predicted to occur via a two-step process involving initial transfer of the inositol phosphate residue to an activated histidine in the active site, with the phosphate intermediate being subsequently subjected to nucleophilic attack by the oxygen of the (phyto)ceramide hydroxyl group resulting in transfer to the sphingoid base. Notably, mutation of His-294 of the yeast IPC synthase, located in the putative active site, results in non-viable haploid cells (24). In support of the phylogenetic analysis in this study the conservation of the catalytic mechanism strongly suggests that fungal, animal and protozoan sphingolipid synthases have evolved into 3 distinct classes from a common ancestor. Conversely, the soluble bacterial SM synthase lacks the motifs believed to be integral for activity of the eukaryotic enzymes (27) indicating that it arose independently.

The data presented here demonstrate that LmIPCS (like its fungal equivalent) is inhibited by Aureobasidin A. Given that IPCS is active in the pathogenic stages of all the parasitic kinetoplastids studied to date (9,22) this demonstrates that the protozoan IPCS represents a tractable drug target. However, a recent study has indicated the T. cruzi enzyme activity is unaffected by this anti-fungal agent (and rustmicin) even at levels 1000 fold higher than that which inhibit the S. cerevisiae enzyme (22). Aureobasidin A acts as a non-competitive inhibitor with respect to ceramide (28) indicating that it does not bind in the catalytic site. However, given the level of sequence conservation outside the active site of the kinetoplastid proteins Aureobasidin A may be expected to significantly inhibit all the identified protozoan IPC synthases. In addition, single amino acid mutations in the conserved catalytic domains of IPC synthase have been shown to be sufficient to confer Aureobasidin A resistance in yeast (5,21,29). None of these mutations are detected in the kinetoplastid sequences and further study is required to ascertain where Aureobasidin A binds and how it inhibits the function of both fungal and protozoan IPC synthases. However, in contrast to the study presented here, the T. cruzi assays were performed on crude parasite membrane extracts (22). Interestingly, similarly assayed samples from fungal Aspergillus spp. are only susceptible to Aureobasidin A in the presence of mammalian multi-drug resistance modulators, indicating that the lack of susceptibility of this important fungal pathogen is due to efflux (30). A similar scenario may prevent Aureobasidin A inhibiting TcIPCS in such assays.

The toxicity and expense of available treatments for leishmaniasis, African sleeping sickness and Chagas” Disease remains a major problem in the treatment of these emerging infectious diseases. This, coupled with the prevalence of drug resistant parasites, makes the discovery of novel drug targets a priority. The identification and characterization of the kinetoplastid IPC synthase, an enzyme with no functional equivalent in mammalian cells, raises the possibility of developing specific inhibitors with little or no mammalian toxicity. In addition, the close evolutionary relationship observed between the kinetoplastid sphingolipid synthases indicates that the development of broad-spectrum inhibitors of this enzyme is a feasible objective, work which may lead to a new generation of anti-protozoals directed against the causative agents of a range of emerging diseases.

Protocol for LmIPCS Activity Assay

The Inositol Phosphoryl Ceramide Synthase is a specific enzyme required for the metabolic pathway needed in the synthesis of the plasma membrane constituents fungi and kinetoplasids (e.g. Leishamanis spp.) whereas its analogue in mammals is the Sphingomyelin Synthase. This fact emphasizes the potentiality of such enzyme as an anti-kinetoplastidae novel drug target.

The protocol outlined below for the activity assay of the LmIPC Synthase can be employed in kinetic studies for that enzyme, in particular in screening assays for identifying agents capable of modulating IPC synthase activity, more particularly identifying agents capable of inhibiting IPC synthase activity.

I. Microsomal Membranes Preparation

Microsomal membranes of S. cerevisiae complemented with LmIPCS were prepared following the procedure described by Fischl et al (32) and Figueiredo et al (33).

S. cerevisiae complemented with LmIPCS were grown up on selective media liquid SD-HIS-URA (24 hrs and 30° C.). Cells were harvested by centrifugation (6900 g, 10 min and 4° C.), washed twice with cold PBS. The precipitated cells (c.a. 8.5 gm wet cell mass in each preparation) were re-suspended in cold STE buffer (25 mM Tris/HCl pH 7.4, 250 mM sucrose and 1 mM EDTA) containing Complete™ protease inhibitor 1 tab/50 ml buffer. Cells were disrupted with glass beads (0.15-0.21 mm, Sigma) using Vortex Mixer (MT20 Chiltern™) for 20 cycles of 1 min each, temperature was kept below 4° C.

The resultant mixture was centrifuged (2800 g, 15 min and 4° C.) to remove unbroken cells, cell-wall debris and glass beads. The supernatant was differentially centrifuged. Stage I (27000 g, 30 min and 4° C.); to remove the granular fraction of cell components. Stage II (150000 g, 90 min and 4° C.); to obtain the fraction enriched in the microsomal membranes. The final pellet was re-suspended in storage buffer (50 mM Tris/HCl pH 7.4, 20% (v/v) glycerol, 5 mM MgCl₂) containing Complete™ protease inhibitor 1 tab/50 ml buffer. Protein concentration was determined using Bradford (34) method (values were referenced to BSA standard curve) and adjusted to 10 mg/ml (Crude membranes).

The crude membranes were washed in 2.5% CHAPS (60 min and <4° C.). The solution was centrifuged (150000 g, 90 min and 4° C.). The precipitated pellet was re-suspended in storage buffer and protein concentration was determined as previous and adjusted to 0.5 mg/ml. Aliquots of 50 μl were stored at −80° C. until use.

II. Fluorescent NBD-C₆-Ceramide LmIPCS Activity Assay

Following the procedure described by Aeed et al (35), 10 parallel assay reactions were run against a Blank control (No microsomal membranes added). A typical assay reaction procedure includes addition of 1 μg of the washed microsomal membranes suspended in 30 μl 71.4 mM potassium phosphate, pH 7.0 to 10 μl solution of 40 μM PI (Bovine liver Phosphatidylinositol Sodium Salt, Avanti Polar Lipids), 20 μM NBD-C₆-Ceramide (Molecular Probes) and 8 mM CHAPS in water (Final volume was 40 μl). The reaction mixture was incubated at 30° C. for the specified period of time and later quenched with 200 μl 99.8% (v/v) MeOH. The reaction product NBD-C₆-IPC was separated from the reaction mixture by adsorbing onto 200 μl (sedimented gel volume) of AG4-X4 (AG4-X4 Resin, Free Base Form, Bio-Rad Laboratories Ltd.), prepared in formate form (36), in a 96-well filter plate using a vacuum manifold. The, reaction-mixture-mounted resin was washed with 5×200 μl of 99.8% (v/v) MeOH, and the product was eluted with 2×100 of 1 M potassium formate in 99.8% (v/v) MeOH. The product was quantified in a fluorescence plate reader (black plates were used to minimize background emission, Perkin Elmer) using 466 nm excitation λ and measuring emission at 536 nm. The 10 reactions covered the time range of 0, 5, 10, 15, 20, 30, 40, 50, 60 and 70 min.

Results and Discussion

The preliminary results obtained from the reaction series lead to the activity curve shown in (FIG. 16). The curve shape is almost the expected shape for an enzyme activity vs. time profile.

The adopted protocol was confirmed to be valid and providing a reliable basis for the Activity Assay of LmIPCS which can be used in further studies to determine the Enzyme Kₘ and Vₘₐₓ values.

The reader's attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings), may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

### REFERENCES

1. McConville, M. J., and Bacic, A. (1989) J Biol Chem 264(2), 757-766
2. Salto, M. L., Bertello, L. E., Vieira, M., Docampo, R., Moreno, S. N., and de Lederkremer, R. M. (2003) Eukaryot Cell 2(4), 756-768
3. Bagnat, M., Keranen, S., Shevchenko, A., and Simons, K. (2000) Proc Natl Acad Sci USA 97(7), 3254-3259
4. Bromley, P. E., Li, Y. O., Murphy, S. M., Sumner, C. M., and Lynch, D. V. (2003) Arch Biochem Biophys 417(2), 219-226
5. Heidler, S. A., and Radding, J. A. (1995) Antimicrob Agents Chemother 39(12), 2765-2769
6. Nagiec, M. M., Nagiec, E. E., Baltisberger, J. A., Wells, G. B., Lester, R. L., and Dickson, R. C. (1997) J Biol Chem 272(15), 9809-9817
7. Georgopapadakou, N. H. (2000) Expert Opin Investig Drugs 9(8), 1787-1796
8. Denny, P. W., Goulding, D., Ferguson, M. A., and Smith, D. F. (2004) Mol Microbiol 52(2), 313-327
9. Zhang, K., Hsu, F. F., Scott, D. A., Docampo, R., Turk, J., and Beverley, S. M. (2005) Mol Microbiol 55(5), 1566-1578
10. Ghosh, S., Bhattacharyya, S., Sirkar, M., Sa, G. S., Das, T., Majumdar, D., Roy, S., and Majumdar, S. (2002) Infect Immun 70(12), 6828-6838
11. Luberto, C., Toffaletti, D. L., Wills, E. A., Tucker, S. C., Casadevall, A., Perfect, J. R., Hannun, Y. A., and Del Poeta, M. M. (2001) Genes Dev 15(2), 201-212
12. Johnson, M., and Davis, R. W. (1984) Mol Cell Biol 4, 1440-1448
13. Shams-Eldin, H., Azzouz, N., Blaschke, T., Hubel, A., Kedees, M., and Schwarz, R. T. (2001) Yeast 18, 33-39
14. Shams-Eldin, H., Azzouz, N., Kedees, M. H., Orlean, P., Kinoshita, T., and Schwarz, R. T. (2002) Mol Biochem Parasitol 120(1), 73-81
15. Jeanmougin, F., Thompson, J. D., Gouy, M., Higgins, D. G., and Gibson, T. J. (1998) Trends Biochem Sci 23(10), 403-405
16. Rost, B. (1996) Methods Enzymol 266, 525-539
17. Ralton, J. E., and McConville, M. J. (1998) J Biol Chem 273(7), 4245-4257
18. Denny, P. W., Field, M. C., and Smith, D. F. (2001) FEBS Lett 491(1-2), 148-153
19. Nagiec, M. M., Young, C. L., Zaworski, P. G., and Kobayashi, S. D. (2003) Biochem Biophys Res Commun 307(2), 369-374
20. Huitema, K., van den Dikkenberg, J., Brouwers, J. F., and Holthuis, J. C. (2004) Embo J 23(1), 33-44
21. Hashida-Okado, T., Yasumoto, R., Endo, M., Takesako, K., and Kato, I. (1998) Curr Genet 33(1), 38-45
22. Figueiredo, J. M., Dias, W. B., Mendonca-Previato, L., Previato, J. O., and Heise, N. (2005) Biochem J 387(Pt 2), 519-529
23. Neuwald, A. F. (1997) Protein Sci 6(8), 1764-1767
24. Levine, T. P., Wiggins, C. A., and Munro, S. (2000) Mol Biol Cell 11(7), 2267-2281
25. Zufferey, R., Allen, S., Barron, T., Sullivan, D. R., Denny, P. W., Almeida, I. C., Smith, D. F., Turco, S. J., Ferguson, M. A., and Beverley, S. M. (2003) J Biol Chem
26. Dickson, R. C., and Lester, R. L. (1999) Biochim Biophys Acta 1426, 347-357
27. Luberto, C., Stonehouse, M. J., Collins, E. A., Marchesini, N., El-Bawab, S., Vasil, A. I., Vasil, M. L., and Hannun, Y. A. (2003) J Biol Chem 278(35), 32733-32743
28. Zhong, W., Murphy, D. J., and Georgopapadakou, N. H. (1999) FEBS Lett 463(3), 241-244
29. Hashida-Okado, T., Ogawa, A., Endo, M., Yasumoto, R., Takesako, K., and Kato, I. (1996) Mol Gen Genet 251(2), 236-244
30. Zhong, W., Jeffries, M. W., and Georgopapadakou, N. H. (2000) Antimicrob Agents Chemother 44(3), 651-653
31. Lisman, Q., Pomorski, T., Vogelzangs, C., Urli-Stam, D., de Cocq van Delwijnen, W., and Holthuis, J. C. (2004) J Biol Chem 279(2), 1020-1029;
32. Fischl, A. S.; Liu, Y.; Browdy, A.; Cremesti, A. E. Methods in Enzymology 2000, 311, 123-130.
33. Figueiredo, J. M.; Dias, W. B.; Mendonca-Previato, L.; Previato, J. O.; Heise, N. Biochem. J. 2005, 519-529.
34. Bradford, M. M. Anal. Biochem. 1976, 72, 248.
35. Aeed, P. A.; Sperry, A. E.; Young, C. L.; Nagiec, M. M.; Elhammer, A. P. Biochem. 2004, 43, 8483-8493.
36. Mandala, S. M.; Thornton, R. A.; Rosenbach, M.; Milligan, J.; Garcia-Calvo, M.; Bull, H. G.; and Kurtz, M. B. J. Biol. Chem. 1997, 272, 32709-32714.

1. A screening method for the identification of an agent that has inositol phosphoryceraminde synthase enzyme inhibitory activity comprising the steps of:
i) providing a polypeptide encoded by a nucleic acid molecule selected from the group consisting of:
a) a nucleic acid molecule consisting of the DNA sequence as represented in FIG. 6A, 7A, 8A, 9A, 10A, 11A, 12A, 13A, 14A or 15A;
b) a nucleic acid molecule comprising DNA sequences that hybridize to the sequence identified in (a) above and which encode a polypeptide that is an inositol phosphorylceramide synthase; and
c) a nucleic acid molecule comprising DNA sequences that are degenerate as a result of the genetic code to the DNA sequence defined in (a) and (b) and that encodes a polypeptide that is an inositol phosphorylceramide synthase.
ii) providing at least one candidate agent to be tested;
iii) forming a preparation that is a combination of (i) and (ii) above; and
iv) testing the effect of said agent on the enzyme activity of said inositol phosphoryceraminde synthase.

2. A polypeptide encoded by a nucleic acid molecule selected from the group consisting of:
i) a nucleic acid molecule consisting of the DNA sequence as represented in FIG. 6A, 7A, 8A, 9A, 10A, 11A, 12A, 13A, 14A or 15A;
ii) a nucleic acid molecule comprising DNA sequences that hybridize to the sequence identified in (i) above under stringent hybridization conditions and which encodes a polypeptide that is an inositol phosphorylceramide synthase; and
iii) a nucleic acid molecule comprising DNA sequences that are degenerate as a result of the genetic code to the DNA sequence defined in (i) and (ii), and encodes a polypeptide that is an inositol phosphorylceramide synthase.

3. A polypeptide according to claim 2 wherein said polypeptide comprises the amino acid sequence as shown in FIG. 7B, 8B, 9B, 10B, 11B, 12B, 13B, 14B or 15B, or sequence variant thereof, wherein said variant is modified by deletion, addition or substitution of at least one amino acid residue.

4. A polypeptide according to claim 2 wherein said polypeptide consists of the amino acid sequence shown in FIG. 7B, 8B, 9B, 10B, 11B, 12B, 13B, 14B or 15B.

5. A polypeptide according to claim 2 wherein said nucleic acid molecule has at least 50% sequence identity, over all or part of the length of the nucleic acid, to the nucleic acid as represented by the nucleic acid sequence in FIG. 6A, 7A, 8A, 9A, 10A, 11A, 12A, 13A, 14A or 15A and which encodes a polypeptide that has inositol phosphorylceramide synthase activity.

6. A polypeptide according to claim 2 wherein said polypeptide is a variant polypeptide and comprises the amino acid sequence represented in FIG. 7B, 8B, 9B, 10B, 11B, 12B, 13B, 14B or 15B, which sequence has been modified by deletion, addition or substitution of at least one amino acid residue wherein said modification retains or modifies the inositol phosphorylceramide synthase activity of said polypeptide.

7. A cell wherein said cell is transformed or transfected with a nucleic acid molecule that encodes a polypeptide according to claim 2.

8. A cell according to claim 7 wherein said nucleic acid molecule is part an expression vector.

9. A cell according to claim 7 wherein said cell is selected from the group consisting of; a fungal cell; insect cell; a mammalian cell; a plant cell.

10. A cell according to claim 9 wherein said cell is a fungal cell.

11. A cell according to claim 9 wherein said cell is a yeast cell.

12. A cell according to claim 11 wherein said cell is a Saccharomyces cerevisiae cell.

13. The use of a polypeptide according to claim 2 for the identification of an agent that modulates the activity of an inositol phosphoryceraminde synthase enzyme.

14. The use of a cell according to claim 7 for the identification of an agent that modulates the activity of an inositol phosphoryceraminde synthase enzyme.

15. The use according to claim 13, wherein said modulation is inhibition of an inositol phosphoryceraminde synthase enzyme.

16. A screening method for the identification of an agent that has inositol phosphoryceraminde synthase enzyme inhibitory activity comprising the steps of:
i) providing a polypeptide according to claim 2;
ii) providing at least one candidate agent to be tested;
iii) forming a preparation that is a combination of (i) and (ii) above; and
iv) testing the effect of said agent on the enzyme activity of said inositol phosphoryceraminde synthase.

17. A method according to claim 16 wherein said agent comprises a ceremide moiety.

18. A method according to claim 16 wherein said agent comprises a diacyl glyceride moiety.

19. A method according to claim 16 wherein said agent comprises an inositol moiety.

20. A method according to claim 16 wherein said method is a cell based method and said polypeptide is expressed by a cell according to any one of claims 7 to 12.

21. A method according to claim 20 wherein said cell is in liquid culture medium.

22. A method according to claim 20 wherein said cell is grown in plate culture on solid growth medium.

23. A method according to claim 20 wherein said agent is included in said liquid culture or solid culture medium.

24. A method to determine the ability of an agent to associate with an inositol phosphorylceramide synthase polypeptide comprising the steps of:
i) providing computational means to perform a fitting operation between said agent and a polypeptide according to claim 2; and
ii) analyzing the results of said fitting operation to quantify the association between the agent and the inositol phosphoryceraminde synthase polypeptide.

25. A method according to claim 24 wherein said agent comprises a ceremide moiety.

26. A method according to claim 24 wherein said agent comprises a diacyl glyceride moiety.

27. A method according to claim 24 wherein said agent comprises an inositol moiety.

28. A method according to claim 27 wherein said agent is modified to alter its binding affinity and/or specificity for said polypeptide.

29. A method according to claim 24 wherein said agent is an antagonist for said polypeptide or modified polypeptide.

30. A method for the rational design of mutations in inositol phosphoryceraminde synthase polypeptides comprising the steps of:
i) providing a 3D model of a first polypeptide as represented by the amino acid sequence in FIG. 7B, 8B, 9B, 10B, 11B, 12B, 13B, 14B or 15B;
ii) providing a 3D model of a variant polypeptide wherein said variant polypeptide is a modified sequence variant of said first polypeptide which is modified by addition, deletion or substitution of at least one amino acid residue in FIG. 7B, 8B, 9B, 10B, 11B, 12B, 13B, 14B or 15B;
iii) comparing the effect on the 3D model of said second polypeptide when compared to the 3D model of said first polypeptide; optionally
iv) testing the effect of said modification on the enzyme activity of said second polypeptide when compared to said first polypeptide.

31. The use of an agent that modulates the activity an inositol phosphoryceraminde synthase enzyme in the manufacture of a medicament for the treatment of a protozoan parasitic disease.

32. The use according to claim 31, wherein said protozoan parasitic disease is a kinetoplastodae disease.

33. The use according to claim 31, wherein said inositol phosphoryceraminde synthase enzyme has a polypeptide sequence according to claim 2.

34. The use according to claim 31 wherein said agent is an aureobasidin antibiotic.

35. The use according to claim 31, wherein said agent is an khafrefungin antibiotic.

36. The use according to claim 31, wherein said agent is an rustmicin antibiotic.

37. The use according to claim 31, wherein said protozoan parasitic disease is leishmaniasis, African trypanosomiasis or Chagas' disease.

38. A method to treat leishmaniasis comprising administering an effective amount of at least one antibiotic selected from the group consisting of: aureobasidin, khafrefungin or rustmicin to a subject in need of treatment.

39. A method to treat African trypanosomiasis comprising administering an effective amount of at least one antibiotic selected from the group consisting of: aureobasidin, khafrefungin or rustmicin to a subject in need of treatment.

40. A method to treat Chagas' disease comprising administering an effective amount of at least one antibiotic selected from the group consisting of: aureobasidin, khafrefungin or rustmicin to a subject in need of treatment.

41. A method according to claim 38 wherein said subject is a human.

42. The use of an agent identified by a method according to claim 16 that modulates the activity an inositol phosphoryceraminde synthase enzyme in the manufacture of a medicament for the treatment of a protozoan parasitic disease.
